(19) United States
(12) Patent Application Publication
(10) Pub. No.: US 2020/0316213 A1
(43) Pub. Date: Oct. 8, 2020

(54) MACROMOLECULAR PLATFORM FOR TARGETING SCAVENGER RECEPTOR A1

(71) Applicant: THE UNITED STATES OF AMERICA, AS REPRESENTED BY THE SECRETARY,DEPARTMENT OF HEALTH AND HUMAN SERVICS, Bethesda, MD (US)

(72) Inventors: David Michael Stevens, Frederick, MD (US); Stephan Timothy Stern, Frederick, MD (US); Scott McNeil, Vienna, VA (US); Marina A. Dobrovolskaia, Frederick, MD (US)

(21) Appl. No.: 16/756,259

(22) PCT Filed: Oct. 15, 2018
(86) PCT No.: PCT/US2018/055794

§ 371 Date: Apr. 15, 2020

Related U.S. Application Data
(60) Provisional application No. 62/572,733, filed on Oct. 16, 2017.

(51) Int. Cl. A61K 47/64; A61K 47/38

(52) U.S. Cl. CPC A61K 47/645; A61K 45/06; A61K 47/38

(57) ABSTRACT

The present invention is directed to a polymer platform comprising poly(L-lysine succinylated) which specifically targets scavenger receptor A1. This platform may be used to conjugate different types of drugs to the polymer for treatment of specific diseases or conditions in a patient. The resulting conjugates display moderate stability or controlled drug release of about 3-80 hours in plasma, and allow delivery and release of drugs and other therapeutic moieties to tissues/cells that express scavenger receptor A1 in a controlled manner.

### CROSS-REFERENCE TO RELATED APPLICATION

This application claims priority to and the benefit of U.S. Provisional Application No. 62/572,733 filed on Oct. 16, 2017, which is hereby incorporated by reference in its entirety.

### BACKGROUND OF THE INVENTION

### 1. Field of the Invention

The present invention is directed to drug delivery platforms, and more specifically to a completely succinylated polymer platform that inherently targets scavenger receptor A1 to deliver drug compounds with great specificity.

### 2. Brief Description of the Related Art

Drug delivery platforms are instruments for selectively delivering a therapeutically active molecular component to target cells. Drug delivery technologies have long claimed the ability to selectively deliver therapeutic cargo to target cells in what is often termed targeted drug delivery. Targeted drug delivery is a method of delivering medication to a patient in a manner that increases the concentration of the medication in some parts of the body relative to others. Typically, nanoparticles would be loaded with drugs and targeted to specific parts of the body where there is solely diseased tissue, thereby avoiding interaction with healthy tissue. The goal of such a system is to prolong, localize, target and have a protracted drug interaction with the diseased tissue. A targeted system offers several advantages, including reduction in the frequency of the dosages taken by the patient, having a more uniform effects of the drug, reduction of drug side-effects, and reduced fluctuation in circulating drug levels. However, despite recent breakthroughs in nanomedicine and drug delivery system technology, there is currently no single targeted nanoscale delivery methodology on the market.

Scavenger receptors are cell surface receptors that are structurally diverse but they typically recognize many different ligands to participate in diverse biological functions. The functional mechanisms of scavenger receptors include endocytosis, phagocytosis, adhesion and signaling, which ultimately leads to the removal of non-self or altered-self targets. Scavenger Receptor A1 (SR-A1, also known as also known as SCARA1, CD204 or macrophage scavenger receptor 1) was initially identified by its ability to mediate the formation of foam cells, a characteristic component of atherosclerotic lesions (Goldstein et al., 1979; Kodama et al., 1990; Krieger and Herz, 1994; Bowdish and Gordon, 2009). However, more recently, a role beyond the handling of cholesterol is emerging for SR-A1 in the pathogenesis of cardiovascular diseases. Experiments have shown that SR-A1 not only functions as a phagocytic receptor and an innate immune recognition receptor, but also plays an important role in cell apoptosis and cell proliferation. These receptor characteristics, and myeloid and endothelial expression, make SR-A1 a useful target for treatment of a variety of conditions, such as cancer, infectious disease, and neurodegenerative and inflammatory conditions.

Poly(lysine succinylated) has been reported as a potential vehicle for delivery of therapeutically active molecular components. International Patent Application Publication WO94/17829 discloses a method of directing the biodistribution of a small molecule by use of macromolecular polymers in a diagnostic or therapeutic protocol for the treatment of a mammalian recipient. The method includes, among other steps, administering to the recipient a conjugate including a directed biodistribution molecule made from a succinylated polylysine polymer and a diagnostically or therapeutically active small molecule agent, in which the succinyl group is used as a common attachment linker, not a targeting ligand. The publication is focused on distribution to renal excretion only, and there is no indication that the directed biodistribution molecule possesses controlled release properties. A prodrug in which a biotin molecule is conjugated to the epsilon (ε)-amino groups of polylysine through an amide group (—C(O)NH—) is disclosed as a specific example. U.S. Pat. No. 6,441,025 to Li et al. discloses water soluble compositions of paclitaxel and docetaxel formed by conjugating the paclitaxel or docetaxel to a water soluble polymer such as poly-glutamic acid, poly-aspartic acid, or poly-lysine, as well as methods of using the compositions for treatment of tumors, auto-immune disorder, or in coating of implantable stents. However, neither of these references disclose use of poly(lysine succinylated) as a drug delivery platform that targets scavenger receptor A1.

What is needed in the art is an improved drug delivery platform that can treat diseases and conditions by targeting scavenger receptor A1. The present invention is believed to be an answer to that need.

### SUMMARY OF THE INVENTION

In an embodiment, a method for delivery of a therapeutically active molecule to a patient through targeting scavenger A1 receptor is provided. The method includes the steps of providing a composition including a conjugate of poly(lysine succinylated) and a therapeutically active molecule, and administering the composition to a patient, wherein the conjugate displays affinity for scavenger A1 receptor.

In another embodiment, a composition for delivery of a therapeutically active molecule by way of targeting to scavenger A1 receptor to a patient is provided. The composition includes a conjugate of poly(lysine succinylated) and a therapeutically active molecule.

These and other aspects of the present invention are described in more detail below.

### BRIEF DESCRIPTION OF THE DRAWINGS

The above and other aspects and features of the present disclosure will become more apparent in the following detailed description when taken in conjunction with reference to the accompanying drawings, in which:

FIG. 1 is a diagram showing a one-step synthesis of polymer-488 using AlexaFluor 488 with poly(lysine succinylated) via EDC HCl and Sulfo-NHS chemistry to form a stable amide bond;

FIG. 2 is a diagram showing flow cytometry data for untreated and polymer-488 treated RAW 264.7 and clone ½ cells;

FIG. 3 is a graph of fluorescence (arbitrary units, a. u.) versus concentration of polymer-488 (milligram per milliliter, mg/mL) illustrating fluorescence data from competitive inhibition study;

FIG. 4 is a graph of fluorescence normalized to inhibitor-free control (percent “%” control) versus inhibitor concentration (milligram per milliliter, mg/mL) illustrating fluorescence data from competitive inhibition study;

FIG. 5 is a graph of fluorescence (percent “%” control) versus competitor concentration (milligram per milliliter, mg/mL) illustrating fluorescence data from competitive binding study between poly(lysine succinylated) (100% succinylated) and 94% succinylated poly(lysine), wherein polymer-488 excitation/emission is 493/516 nm, data are expressed as mean±SD (n=3), and unpaired t-test p<0.05;

FIG. 6 shows representative whole-body images of Balb/c mice treated with Cy7.5 labelled poly(lysine succinylated) via tail vein injection;

FIG. 7 shows representative organ images of Balb/c mice treated with Cy7.5 labelled poly(lysine succinylated) via tail vein injection (V=ventral side and D=dorsal side);

FIG. 8 shows average organ distribution after tail vein injection, wherein the dashed line represents typical background level;

FIG. 9 shows representative whole-body images of Balb/c mice treated with Cy7.5 labelled poly(lysine succinylated) via intraperitoneal injection;

FIG. 10 shows representative organ images of Balb/c mice treated with Cy7.5 labelled poly(lysine succinylated) via intraperitoneal injection (V=ventral side and D=dorsal side);

FIG. 11 shows average organ distribution after intraperitoneal injection, wherein the dashed line represents typical background level;

FIG. 12 is a diagram showing anti-alexa-488 staining of fixed tissues following IV or ID administration of polymer-488;

FIG. 13 is a diagram showing non-alcohol-containing drugs conjugated using a multi-step synthesis;

FIG. 14 is a ¹H NMR spectrum of allyl-functionalized poly(L-lysine succinylated) in D₂O;

FIG. 15 is a diagram showing a one-step synthetic route to conjugate paclitaxel to poly(L-lysine succinylated) through diisopropylcarbodiimide (DIC) coupling;

FIG. 16 is a graph of released paclitaxel (percent total paclitaxel concentration) versus time (hours, h) in human plasma or PBS (supplemented with 1% Tween-80 by volume, pH 7.4) normalized to free paclitaxel controls, illustrating normalized drug-release profiles of paclitaxel released from prodrug, according to an embodiment of the present invention, at 37° C. over the course of 24 hours;

FIG. 17A is a graph of concentration of released paclitaxel (PTX) (nanograms per milliliter, ng/mL) versus time (hours, h) illustrating a pharmacokinetic profile of commercial Abraxane versus prodrug-released paclitaxel, according to an embodiment of the present invention, each group being dosed at 5 milligrams per kilogram (mg/kg);

FIG. 17B is a graph of concentration of released paclitaxel (PTX) (nanograms per milliliter, ng/mL) versus time (hours, h) illustrating a pharmacokinetic profile of total versus released paclitaxel from prodrug dosed at 5 milligrams per kilogram (mg/kg);

FIG. 18 is a diagram showing a one-step synthetic route to conjugate lamivudine to poly(L-lysine succinylated) through diisopropylcarbodiimide (DIC) coupling;

FIG. 19 is a graph of concentration of released lamivudine [Drug] (micrograms per milliliter, μg/mL) versus time (hours, h) in human plasma, illustrating a drug-release profile of released lamivudine from prodrug, according to an embodiment of the present invention, at 37° C. up to 24 hours;

FIG. 20 is a diagram showing a one-step synthetic route to conjugate emtricitabine to poly(L-lysine succinylated) through diisopropylcarbodiimide (DIC) coupling;

FIG. 21 is a graph of % (percent) drug release versus time (hours, h) showing in vitro drug release of the emtricitabine prodrug in human plasma at 37° C. over 23 hours (T_{1/2}˜10 hours);

FIG. 22 is a graph of % (percent) drug release versus time (hours, h) showing in vitro drug release of the emtricitabine prodrug in PBS (pH 7.4) at 37° C. over 48 hours (T_{1/2}˜67 hours);

FIG. 23 is a graph of concentration (nanograms per milliliter, ng/mL) versus time (hours, h) showing emtricitabine concentrations in plasma of prodrug and emtricitabine control after IV bolus dose at 10 mg/kg in male Sprague-Dawley rats;

FIG. 24 is a diagram showing a one-step synthetic route to conjugate PI3K/mTOR dual inhibitor drug PI-103 to poly(L-lysine succinylated) through diisopropylcarbodiimide (DIC) coupling;

FIG. 25 is a graph of % (percent) drug release versus time (hours, h) showing in vitro drug release of the P1-103 prodrug in human plasma at 37° C. over 24 hours;

FIG. 26 is a graph of % (percent) drug release versus time (hours, h) showing in vitro drug release of the PI-103 prodrug in PBS (pH 7.4, 1% Tween-80 v/v) at 37° C. over 24 hours (T_{1/2}˜40 hours);

FIG. 27 is a diagram showing the IHC analysis of resected melanoma tumors in mice treated with saline, polymer control, or PI-103 prodrug;

FIG. 28 is a diagram showing iNOS:CD206 ratio (M1:M2 markers, respectively) for each treatment group, wherein the black circles represent individual iNOS:CD206 values within each group, grey circles represent the average iNOS:CD206 ratio for each group; and

FIG. 29 is a graph of tumor volume (cubic millimeters, mm³) versus study days showing tumor volume in syngeneic B16-F10 melanoma Balb/c model following treatments with PI-103 prodrug (0.1, 1, 10 mg/kg PI-103 equivalent, 10 mL/kg), polymer blank, and saline controls, wherein the treatments were administered via tail-vein injection every other day for a total of 5 injections.

### DETAILED DESCRIPTION OF THE INVENTION

### Terminology

Compounds are described using standard nomenclature. Unless defined otherwise, all technical and scientific terms used herein have the same meaning as is commonly understood by one of skill in the art to which this invention belongs.

The terms “a” and “an” do not denote a limitation of quantity, but rather denote the presence of at least one of the referenced items. The term “or” means “and/or”. The terms “comprising,” “having,” “including,” and “containing” are to be construed as open-ended terms (i.e., meaning “including, but not limited to”).

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art of this disclosure.

Furthermore, the disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

All compounds are understood to include all possible isotopes of atoms occurring in the compounds. Isotopes include those atoms having the same atomic number but different mass numbers and encompass heavy isotopes and radioactive isotopes. By way of general example, and without limitation, isotopes of hydrogen include tritium and deuterium, and isotopes of carbon include ¹¹C, ¹³C, and ¹⁴C. Accordingly, the compounds disclosed herein may include heavy or radioactive isotopes in the structure of the compounds or as substituents attached thereto. Examples of useful heavy or radioactive isotopes include ¹⁸F, ¹⁵N, ¹⁸O, ⁷⁶Br, ¹²⁵I and ¹³¹I.

The opened ended term “comprising” includes the intermediate and closed terms “consisting essentially of” and “consisting of.”

A dash (“-”) that is not between two letters or symbols is used to indicate a point of attachment for a substituent.

“Conjugate” means a chemical entity, in which two or more compounds are bonded to each other through a coordination, covalent, or ionic bond.

“Pharmaceutical compositions” means compositions comprising at least one active agent, such as a compound or salt of Formula 3, and at least one other substance, such as a carrier. Pharmaceutical compositions meet the U.S. FDA's GMP (good manufacturing practice) standards for human or non-human drugs.

A “patient” means a human or non-human animal in need of medical treatment. Medical treatment can include treatment of an existing condition, such as a disease or disorder or diagnostic treatment. In some embodiments the patient is a human patient.

“Providing” means giving, administering, selling, distributing, transferring (for profit or not), manufacturing, compounding, or dispensing.

“Treatment” or “treating” means providing an active compound to a patient in an amount sufficient to measurably reduce any disease symptom, slow disease progression or cause disease regression. In certain embodiments treatment of the disease may be commenced before the patient presents symptoms of the disease.

A “physiologically effective amount” of a pharmaceutical composition means an amount effective, when administered to a patient, to provide a therapeutic benefit such as an amelioration of symptoms, decrease disease progression, or cause disease regression.

A “therapeutically active molecule” means a compound which can be used for diagnosis or treatment of a disease. The compounds can be small molecules, peptides, proteins, or other kinds of molecules.

A significant change is any detectable change that is statistically significant in a standard parametric test of statistical significance such as Student's T-test, where p<0.05.

### Embodiments

The present invention is directed to a succinylated polymer conjugate that inherently targets scavenger receptor A1 to deliver drug compounds with great control and specificity, and a method of delivering therapeutically active molecules to specific targets in a patient using the succinylated polymer conjugate. The conjugate is based on the anionic polymer poly(L-lysine succinylated), which itself displays high affinity for the scavenger receptor A1 and does not require attachment of any ligands specifically targeting the receptor. The conjugate includes a succinyl moiety bonded to the ε-amino group of L-lysine, wherein the succinyl moiety includes a pendant carboxylic acid group capable of conjugating to a drug molecule through a hydrolyzable ester bond. As will be discussed below, various drug molecules may be attached to the carboxylic acid group of poly(L-lysine succinylated) to form a poly(L-lysine succinylated) conjugate. A poly(L-lysine succinylated) conjugate, as used herein, is therefore defined as a chemical entity in which a therapeutically active molecule is bonded to the poly(L-lysine succinylated) through an ester bond. Such a poly(L-lysine succinylated) conjugate may find utility in a variety of applications including drug delivery to the tissues expressing scavenger receptor A1 (such as liver), treatment of lymphoid/macrophage HIV reservoirs, targeting of tumor associated macrophage, among others. Each of the components of the poly(L-lysine succinylated) conjugate is described in more detail below.

As used herein, the term “poly(lysine succinylated)” refers to a polymer having the following structure:

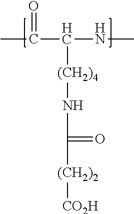

Poly(lysine succinylated) may be prepared, for example, by succinylation of poly-L-lysine with succinic anhydride in the presence of a base. As a result of the reaction, some or all of the primary amino groups become succinylated, including terminal and ε-amino groups. Succinylation of some of the amino groups of poly-L-lysine results in a partially succinylated poly-L-lysine. Succinylation of all or substantially all amino groups of poly-L-lysine provides a completely succinylated poly-L-lysine. As used herein, the term “succinylation of substantially all amino groups” refers to succinylation of amino groups, present in poly-L-lysine, in an amount of 99% or greater, for example, 99.5% or greater, or 99.9% or greater. Therefore, the degree of succinylation in the completely succinylated poly-L-lysine may be 99% or greater, for example, 99.5% or greater, or 99.9% or greater.

The molecules of poly(lysine succinylated) include carboxylic acid groups, which are capable of reacting with compounds having hydroxyl groups, such as alcohols or phenols, to produce esters. Accordingly, various hydroxyl containing molecules B-OH can be attached by way of an ester linkage to poly(lysine succinylated) to form a conjugate. The attachment may be schematically represented as follows:

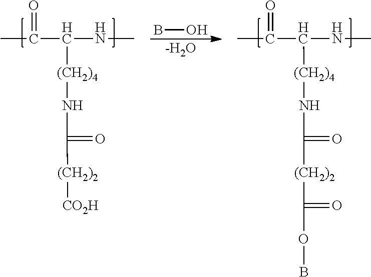

In an embodiment, B—OH may be a therapeutically active molecule capable of producing a biological effect. For example, the therapeutically active molecule may be a drug molecule useful for treatment of a disease or condition selected from acne, attention deficit/hyperactivity disorder (ADHD), human immunodeficiency virus (HIV), Rift Valley fever virus, allergies, Alzheimer's disease, angina, anxiety, arthritis, asthma, bipolar disorder, bronchitis, cancer, elevated cholesterol problems, cold and flu, constipation, chronic obstructive pulmonary disease (COPD), depression, type 1 and 2 diabetes, diarrhea, eczema, erectile dysfunction, fibromyalgia, gastrointestinal disorders, gastroesophageal reflux disease (GERD), gout, hair loss, hay fever, heart disease, hepatitis A, hepatitis B, hepatitis C, hypertension, hypothyroidism, incontinence, irritable bowel syndrome, insomnia, menopause, mental health, migraine, osteoarthritis, osteoporosis, pain, psoriasis, rheumatoid arthritis, schizophrenia, seizures, sexually transmitted disorder (STD), stroke, swine flu, urinary tract infection (UTI), weight loss, but are not limited thereto.

In an embodiment, the hydroxyl group containing molecule B-OH may be a small molecule drug, a peptide, or a vaccine. The inventors of the present invention have found that the poly(L-lysine succinylated) may conjugate different types of drugs or other moieties to the polymer to achieve a moderately stable (i.e., controlled) release of a therapeutic component. Because of its high affinity for scavenger receptor A1, poly(L-lysine succinylated) may thus serve as a convenient platform to deliver various therapeutically active molecules to tissues/cells that express scavenger receptor A1.

In a preferred embodiment, the therapeutically active molecule may be an anti-cancer drug such as paclitaxel or an anti-viral drug such as lamivudine. Both paclitaxel and lamivudine are particularly suitable for the compounds and methods of the present invention because each contain hydroxyl groups that may be attached to poly(L-lysine succinylated) through an ester linkage —C(═O)O—. Specific examples of therapeutic formulations, including paclitaxel (as a model chemotherapeutic) and lamivudine (as a model anti-HIV drug), have been developed and are described below. The paclitaxel prodrug was found to have a drug half-life of 40 hours in plasma and demonstrated a similar 40 hour release half-life during in vivo pharmacokinetics study in rats. The prodrug also showed specificity of almost 100% to the macrophage cell lines containing the receptor. Other examples of suitable therapeutic compounds useful in the present invention may include gemcitabine (as another model chemotherapeutic), rapamycin (as an anti-viral or anti-cancer drug), and everolimus (as an analog of rapamycin).

The amount of the therapeutically active molecule B-OH in the poly(lysine succinylated) conjugate may be about 1% or greater based on the total weight of the poly(lysine succinylated) conjugate. For example, the amount of the therapeutically active molecule in the poly(lysine succinylated) conjugate may be about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, or about 75%, or greater, based on the total weight of the conjugate.

The number of the therapeutically active molecules conjugated per molecule of poly(lysine succinylated) may be about 1, about 2, about 3, about 4, about 5, about 6, about 7, about 8, about 9, about 10, about 15, about 20, about 25, about 30, about 35, about 40, about 45, about 50, about 55, about 60, about 65, about 70, about 75, or greater.

The amount of the completely poly(lysine succinylated) polymer portion in the conjugate may be about 25% or greater based on the total weight of the conjugate. For example, the amount of the completely poly(lysine succinylated) polymer portion in the conjugate may be about 25%, about 30%, about 35%, about 40%, about 45%, about 50%, about 55%, about 60%, about 65%, about 70%, about 75%, about 80%, about 90%, or about 95%, or greater, based on the total weight of the conjugate.

As noted above, poly(L-lysine succinylated) may be either partially or completely succinylated. A complete succinylation results in substantially 100% conversion of all primary amino groups to succinate groups which are necessary for conjugation of drugs through esterification. Because the succinylated groups also act as targeting ligands, a complete succinylation of the poly-L-lysine offers a number of advantages such as increased targeting of scavenger receptor A1 and maximization of drug loading. The complete succinylation provides the maximum number of succinylated sites on the polymer, which allows for high drug loading while still having available pendant succinate groups that are necessary for targeting scavenger receptor A1. In contrast, a partial succinylation results in less than 100% conversion of all primary amines to succinate groups, with unmodified amino groups being present in the polymer. Since the unmodified amino groups may interfere with subsequent conjugation reactions of the drug to the polymer, they must be protected by a reaction with a capping agent, such as acetic anhydride. Thus, the use of a partially succinylated poly-L-lysine results in a decreased number of succinylated sites on the polymer, reduced targeting capacity, and decreased drug loading.

The composition including a conjugate, according to an embodiment of the present invention, has controlled drug release properties. Most formulations known in the prior art (prodrugs, micelles, nanoparticles, liposomes) are either very stable (i.e., release the drug too slowly to achieve efficacy) or unstable (i.e., release most or all drug immediately or within an hour of dilution in plasma). With regard to the prior art formulations, it is not uncommon to use the term “controlled release” or similar phrases. However, more often than not, researchers are evaluating drug release formulations in vitro using either non-optimal conditions or non-physiological media. Most drug release assays reported in the prior art use phosphate-buffered saline (PBS) as a release media. Nonetheless, the prior art formulations that appear to be stable and release the drug slowly in PBS, dissociate immediately when placed into plasma. In contrast, the inventors of the present invention discovered new poly(L-lysine succinylated) prodrugs having a drug release half-life of 3-80 hours, for example, 10-50 hours in plasma. The prodrugs, according to an embodiment of the present invention, also demonstrate about the same release half-lifein rats. Thus, the controlled drug release properties are seen both in plasma and in rats.

While the scavenger receptor A1 has a number of reported ligands, to prepare a prodrug, most research groups use a known ligand or inhibitor of the receptor to conjugate it to a nanoparticle or polymer in order to increase affinity of the formulation for the receptor. In contrast, the poly(L-lysine succinylated) prodrug, according to an embodiment of the present invention, shows itself high affinity for the receptor through succinylated amino-groups, and does not need to be conjugated to any additional targeting ligands.

The conjugates, according to an embodiment of the present invention, also display remarkable specificity of 100% positive for cells that express scavenger receptor A1, after 24 hours of incubation. While there are multiple mechanisms for particles/formulations to be taken up by cell during this substantial period, it is surprising to see that the polymer does not bind at all to the cells that do not express scavenger receptor A1.

In an embodiment, a conjugate of poly(L-lysine succinylated) and paclitaxel is provided. The conjugate may be obtained by a reaction between poly(L-lysine succinylated) and a paclitaxel molecule. Since the molecule of paclitaxel contains three hydroxyl groups, each of these hydroxyl groups may be attached to the polymer. Depending on the reaction conditions, selective attachment can be carried out. For example, the molecule of paclitaxel can be selectively attached to the polymer through a 2′-hydroxyl group. In other embodiment, the molecule of paclitaxel can be selectively attached to the polymer through a 7-hydroxyl group. In still other embodiment, the molecule of paclitaxel can be selectively attached to the polymer through both 2′-hydroxyl group and 7-hydroxyl group.

In an embodiment, the poly(lysine succinylated) paclitaxel conjugate has the following formula:

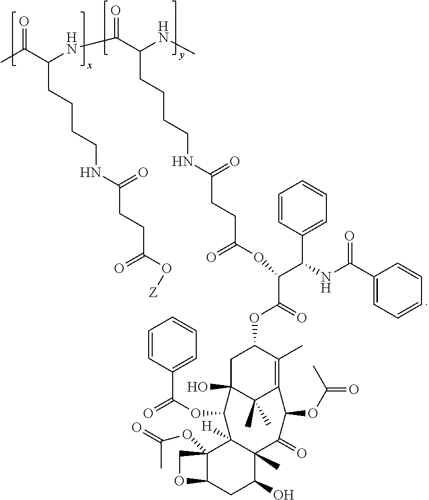

In another embodiment, the poly(lysine succinylated) PI-103 conjugate has the following formula:

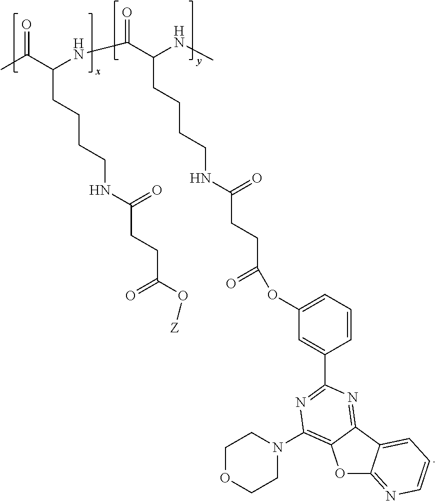

In the above formulae, x and y may be variables selected such that x+y=1, and Z may be H or Na. In the above formula, “x” and “y” represent molar fractions of the corresponding repeating units constituting the conjugate, and “x+y=1” means that the conjugate essentially includes repeating units designated by “x” and “y”, and does not include any other repeating units in substantial quantity (the sum of the molar fractions of the repeating units designated by “x” and “y” adds up to constitute a whole, which is “1”).

For example, y may be an integer between 1 and 10, and x may be (40−y) or (250−y), depending on the length of the polymer.

In an embodiment, a composition including a conjugate of poly(L-lysine succinylated) and lamivudine is provided. The conjugate has the following formula:

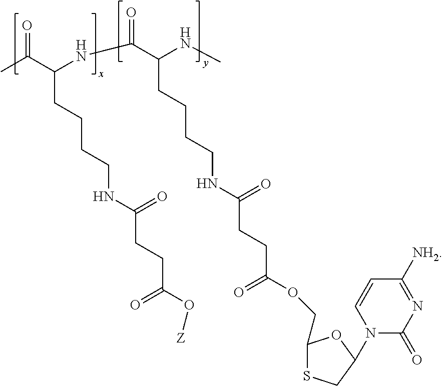

In another embodiment, a composition including a conjugate of poly(L-lysine succinylated) and emtricitabine is provided. The conjugate has the following formula:

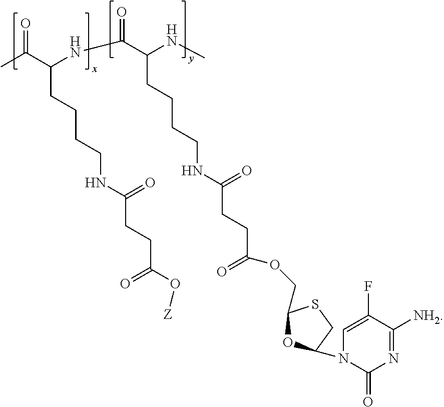

In the above formula, x and y may be variables selected such that x+y=1, and Z may be H or Na.

For example, y may be an integer between 1 and 10, and x may be (40−y) or (250−y), depending on the length of the polymer.

The composition may further include at least one pharmaceutically acceptable excipient. A pharmaceutically acceptable excipient, as used herein, refers to a non-active pharmaceutical ingredient (“API”) substance such as a disintegrator, a binder, a filler, and a lubricant used in formulating pharmaceutical products. Each of these substances is generally safe for administering to humans according to established governmental standards, including those promulgated by the United States Food and Drug Administration (“FDA”).

A disintegrator, as used herein, refers to one or more of agar-agar, algins, calcium carbonate, carboxymethylcellulose, cellulose, clays, colloid silicon dioxide, croscarmellose sodium, crospovidone, gums, magnesium aluminium silicate, methylcellulose, polacrilin potassium, sodium alginate, low substituted hydroxypropylcellulose, and cross-linked polyvinylpyrrolidone hydroxypropylcellulose, sodium starch glycolate, and starch, but is not limited thereto.

A binder, as used herein, refers to one or more of microcrystalline cellulose, hydroxymethyl cellulose, and hydroxypropylcellulose, but is not limited thereto.

A filler, as used herein, refers to one or more of calcium carbonate, calcium phosphate, dibasic calcium phosphate, tribasic calcium sulfate, calcium carboxymethylcellulose, cellulose, dextrin derivatives, dextrin, dextrose, fructose, lactitol, lactose, magnesium carbonate, magnesium oxide, maltitol, maltodextrins, maltose, sorbitol, starch, sucrose, sugar, and xylitol, but is not limited thereto.

A lubricant, as used herein, refers to one or more of agar, calcium stearate, ethyl oleate, ethyl laureate, glycerin, glyceryl palmitostearate, hydrogenated vegetable oil, magnesium oxide, magnesium stearate, mannitol, poloxamer, glycols, sodium benzoate, sodium lauryl sulfate, sodium stearyl, sorbitol, stearic acid, talc, and zinc stearate, but is not limited thereto.

In an embodiment, a method for delivery of a therapeutically active molecule to a patient through targeting scavenger A1 receptor is provided. The method includes the steps of providing a composition including a conjugate of poly(lysine succinylated) and a therapeutically active molecule, as described above, and administering the composition to a patient.

The composition according to the present invention may be administered to a patient by various routes. Examples of routes of administration include, but are not limited to, parenteral, e.g., intravenous, intradermal, subcutaneous, oral, intranasal (e.g., inhalation), transdermal (e.g., topical), transmucosal, and rectal administration. In an embodiment, the composition is formulated in accordance with routine procedures as a pharmaceutical composition adapted for intravenous, subcutaneous, intramuscular, oral, intranasal, or topical administration to human beings. Typically, compositions for intravenous administration are solutions in sterile isotonic aqueous buffer.

In accordance with any of the embodiments, the composition according to the present invention can be administered orally to a subject in need thereof. Formulations suitable for oral administration can consist of (a) liquid solutions, such as an effective amount of the compound dissolved in diluents, such as water, saline, or orange juice and include an additive, such as cyclodextrin (e.g., α-, β-, or γ-cyclodextrin, hydroxypropyl cyclodextrin) or polyethylene glycol (e.g., PEG400); (b) capsules, sachets, tablets, lozenges, and troches, each containing a predetermined amount of the active ingredient, as solids or granules; (c) powders; (d) suspensions in an appropriate liquid; and (e) suitable emulsions and gels. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and cornstarch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such carriers as are known in the art.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The composition according to the present invention can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene-polypropylene copolymers, (d) amphoteric detergents such as, for example, alkyl-beta-aminopropionates, and 2-alkyl-imidazoline quaternary ammonium salts, and (3) mixtures thereof.

The parenteral formulations will typically contain from about 0.5 to about 25% by weight of the composition according to the present invention in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

The composition according to the present invention may be made into injectable formulations. The requirements for effective pharmaceutical carriers for injectable compositions are well known to those of ordinary skill in the art. See Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986).

Topically applied compositions are generally in the form of liquids (e.g., mouthwash), creams, pastes, lotions and gels. Topical administration includes application to the oral mucosa, which includes the oral cavity, oral epithelium, palate, gingival, and the nasal mucosa. In some embodiments, the composition contains at least one active component and a suitable vehicle or carrier. It may also contain other components, such as an anti-irritant. The carrier can be a liquid, solid or semi-solid. In embodiments, the composition is an aqueous solution, such as a mouthwash. Alternatively, the composition can be a dispersion, emulsion, gel, lotion or cream vehicle for the various components. In one embodiment, the primary vehicle is water or a biocompatible solvent that is substantially neutral or that has been rendered substantially neutral. The liquid vehicle can include other materials, such as buffers, alcohols, glycerin, and mineral oils with various emulsifiers or dispersing agents as known in the art to obtain the desired pH, consistency and viscosity. It is possible that the compositions can be produced as solids, such as powders or granules. The solids can be applied directly or dissolved in water or a biocompatible solvent prior to use to form a solution that is substantially neutral or that has been rendered substantially neutral and that can then be applied to the target site. In embodiments of the invention, the vehicle for topical application to the skin can include water, buffered solutions, various alcohols, glycols such as glycerin, lipid materials such as fatty acids, mineral oils, phosphoglycerides, collagen, gelatin and silicone based materials.

The composition according to the present invention, alone or in combination with other suitable components, can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer.

The dose administered to the mammal, particularly human and other mammals, in accordance with the present invention should be sufficient to affect the desired response. One skilled in the art will recognize that dosage will depend upon a variety of factors, including the age, condition or disease state, predisposition to disease, genetic defect or defects, and body weight of the mammal. The size of the dose will also be determined by the route, timing and frequency of administration as well as the existence, nature, and extent of any adverse side-effects that might accompany the administration of a particular composition and the desired effect. It will be appreciated by one of skill in the art that various conditions or disease states may require prolonged treatment involving multiple administrations.

The composition according to the present invention may be administered in an effective amount. An “effective amount” means an amount sufficient to show a meaningful benefit in an individual, e.g., promoting at least one aspect of tumor cell cytotoxicity (e.g., inhibition of growth, inhibiting survival of a cancer cell, reducing proliferation, reducing size and/or mass of a tumor (e.g., solid tumor)) or anti-viral effect, or treatment, healing, prevention, delay of onset, halting, or amelioration of other relevant medical condition(s) associated with a particular cancer or viral infection. The meaningful benefit observed in the patient can be to any suitable degree (10, 20, 30, 40, 50, 60, 70, 80, 90% or more). In some aspects, one or more symptoms of the cancer or viral infection are prevented, reduced, halted, or eliminated subsequent to administration of a composition according to the present invention, thereby effectively treating the disease to at least some degree.

Effective amounts may vary depending upon the biological effect desired in the individual, condition to be treated, and/or the specific characteristics of the composition according to the present invention and the individual. In this respect, any suitable dose of the composition can be administered to the patient (e.g., human), according to the type of disease to be treated. Various general considerations taken into account in determining the “effective amount” are known to those of skill in the art and are described, e.g., in Gilman et al., eds., Goodman And Gilman's: The Pharmacological Bases of Therapeutics, 8th ed., Pergamon Press, 1990; and Remington's Pharmaceutical Sciences, 17th Ed., Mack Publishing Co., Easton, Pa., 1990, each of which is herein incorporated by reference. The dose of the composition according to the present invention desirably comprises about 0.1 mg per kilogram (kg) of the body weight of the patient (mg/kg) to about 400 mg/kg (e.g., about 0.75 mg/kg, about 5 mg/kg, about 30 mg/kg, about 75 mg/kg, about 100 mg/kg, about 200 mg/kg, or about 300 mg/kg). In another embodiment, the dose of the composition according to the present invention comprises about 0.5 mg/kg to about 300 mg/kg (e.g., about 0.75 mg/kg, about 5 mg/kg, about 50 mg/kg, about 100 mg/kg, or about 200 mg/kg), about 10 mg/kg to about 200 mg/kg (e.g., about 25 mg/kg, about 75 mg/kg, or about 150 mg/kg), or about 50 mg/kg to about 100 mg/kg (e.g., about 60 mg/kg, about 70 mg/kg, or about 90 mg/kg).

The present disclosure is illustrated and further described in more detail with reference to the following non-limiting examples.

### EXAMPLES

### Example 1. Poly(L-Lysine Succinylated) for Scavenger Receptor A1 Targeting

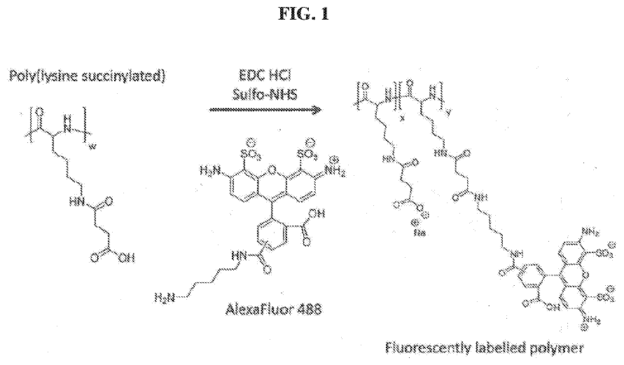

Using EDC (1-ethyl-3-(3-dimethylaminopropyl)carbodiimide) coupling, AlexFluor 488 fluorescent dye was attached to the polymer through stable amide bonds (hereinafter “polymer-488”) (FIG. 1).

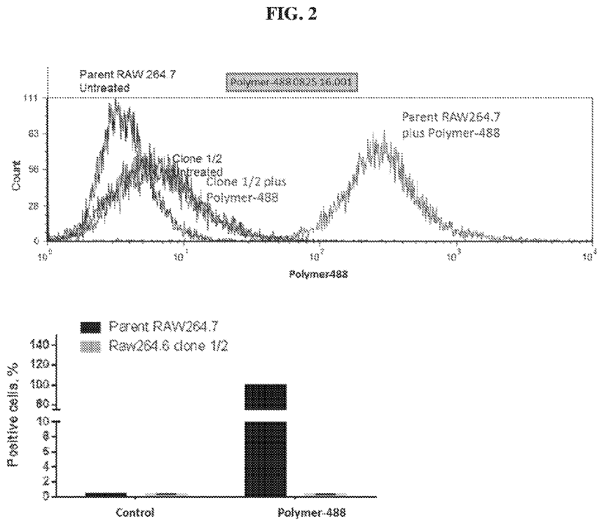

Interactions with scavenger receptor A1 were validated in the Raw 264.7 derivative cells (Clone ½) which do not express scavenger receptor A1. Both the parent cells and the SR-A deficient Clone ½ were treated with the polymer-488 at a concentration of 0.001 mg/ml for 24 hours and analyzed by flow cytometry. Untreated cells were used as control. The flow cytometry results show uptake of the polymer-488 by all cells in the parent Raw 264.7 population and virtually no uptake by the clone ½ cells (FIG. 2).

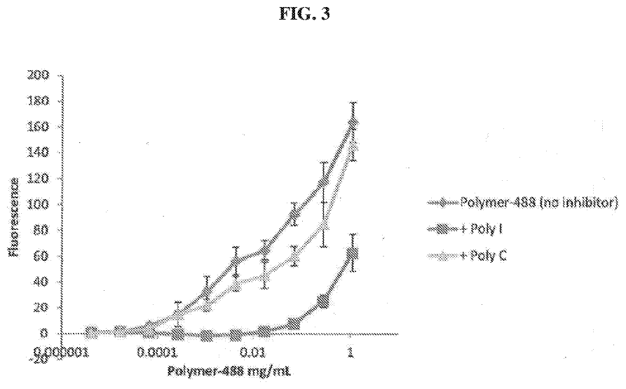

The fluorescently labelled poly(L-lysine succinylated) was also tested for cell uptake in a macrophage cell line Raw 264.7 in the presence of competitive inhibitors and relevant controls. In this experiment, Raw 264.7 macrophages were treated with various concentrations of polymer-488 with either polyinosinic acid (poly I, known inhibitor for scavenger receptor A), polycytidylic acid (poly C, negative control, which does not inhibit scavenger receptor A), or no inhibitor. The results shown in FIG. 3 indicate inhibition of scavenger receptor interaction in presence of poly I and not with poly C, which is consistent with SRA-specific interactions. At higher concentrations of polymer-488, fluorescence is seen due to competing off the poly I.

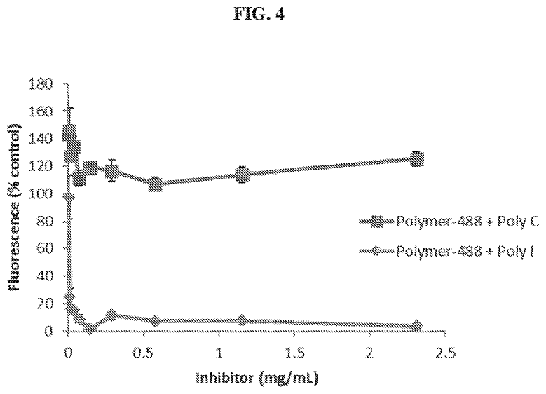

In a follow up study, cells were treated with various concentrations of either poly I or poly C while polymer-488 concentration remained constant. In this study, Raw 264.7 cells were incubated overnight at 400,000 cells/mL. The cells were treated with various concentrations of polymer-488 and either 200 μg/mL poly I, 200 μg/mL poly C, or no inhibitor. The cells were incubated at 37° C. for 3 hours, washed 3 times with media, and fluorescence measured. The results shown in FIG. 4 indicate virtually no inhibition of fluorescence in the poly C group while there is a dose-dependent inhibition in the poly I treated groups. These results further validate the specific interaction between poly(lysine succinylated) and scavenger receptor A1.

Taken together, the results shown in FIGS. 2-4 indicate a strong interaction between the poly(L-lysine succinylated) and scavenger receptor A1. The polymer appears to have a remarkable ability to be taken up by cells that express receptor A1, and therefore, could be used as a targeted drug delivery system to the cells that express this receptor, particularly macrophages and other myeloid cells.

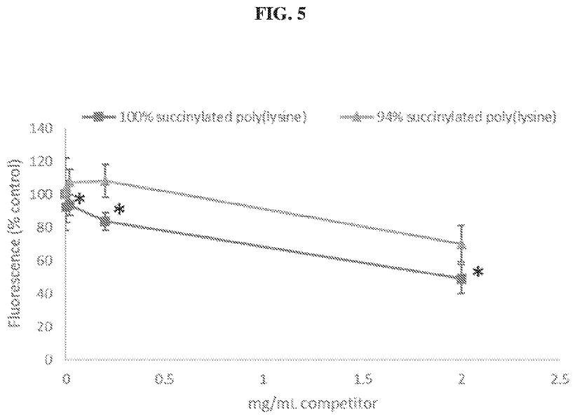

In a follow-up competitive binding study, the binding of poly(L-lysine succinylated), which is 100% succinylated, to Raw 264.7 cells was compared to that of a partially succinylated poly(lysine) to determine if the degree polymer succinylation affects interaction with scavenger receptor A1 on the cell surface. The partially succinylated poly(lysine) was synthesized by reacting poly(L-lysine) (Mₙ=41,000) with succinic anhydride in carbonate buffer followed by acetic anhydride addition. ¹H NMR confirmed the polymer was partially succinylated (94%) and the remaining primary amine groups were capped by acetylation. Raw 264.7 cells were treated with 5 μg/mL fluorescent polymer-488 and various concentrations of fully succinylated (100%) poly(lysine) and partially succinylated (94%) poly(lysine). The cells were incubated at 37° C. for 3 hours, washed 3 times with media, and fluorescence measured. The results displayed in FIG. 5 show dose-dependent decreases in fluorescence when competing with either the 100% or 94% succinylated polymers. Unexpectedly, however, the degree of binding inhibition was dramatically greater for the 100% succinylated polymer in comparison to the 94% succinylated polymer. Since the degree of polymer succinylation was similar for the two constructs (100% vs. 94%), it was unanticipated to see such a large difference in scavenger receptor A1 competition. This unforeseen result shows that even minor differences in the degree of polymer succinylation (i.e., 100% vs. 94%) can have significant effects on receptor interaction, and for this reason, the 100% succinylated poly(lysine) polymer was chosen as the lead prodrug platform over partially succinylated poly(lysine) polymer.

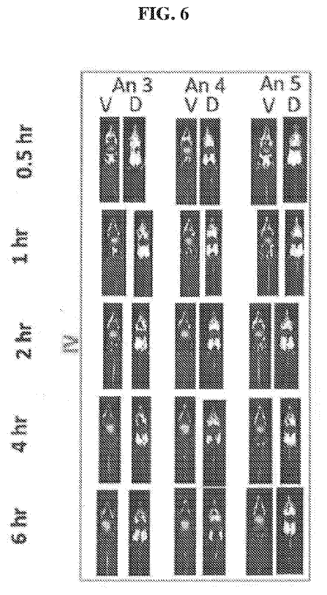

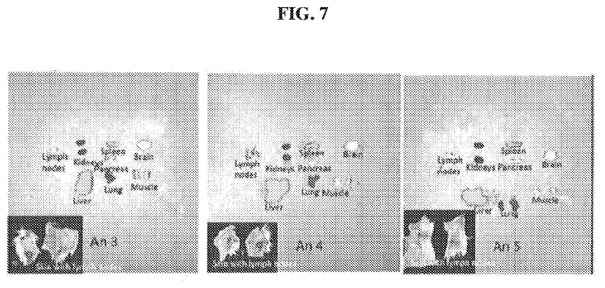

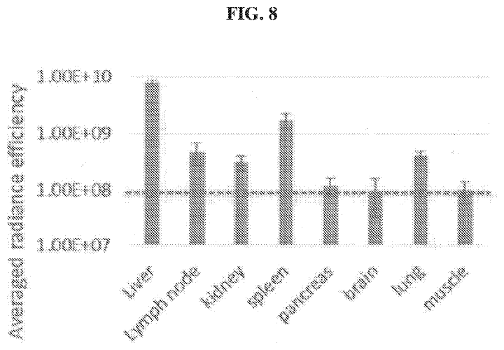

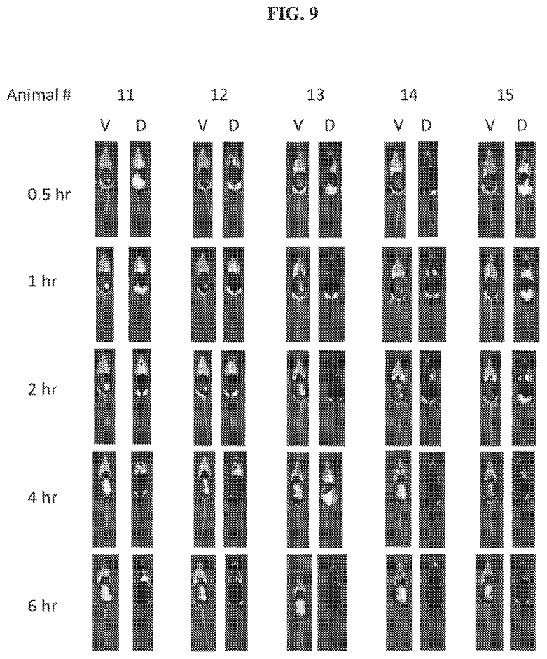

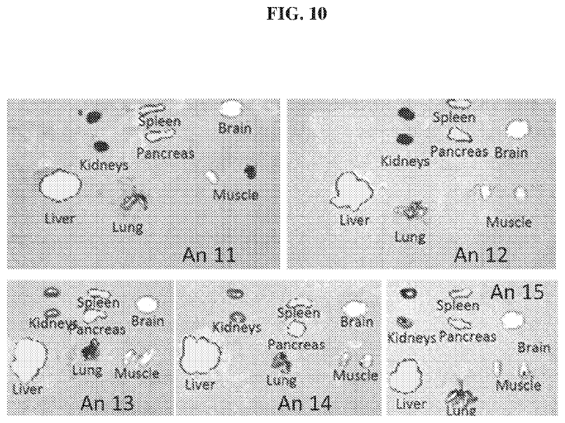

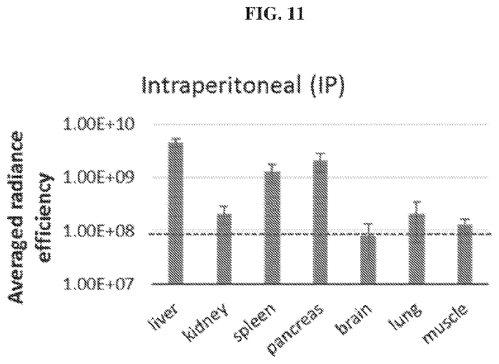

Biodistribution of poly(lysine succinylated) was assessed in mice. The polymer was labelled with Cyanine7.5 amine near-IR dye and administered via tail vein injection or intraperitoneal injection. At various time points, whole-body images were taken, and organs were harvested after 6 hours and imaged. Representative images are shown in FIGS. 6-11. As expected, the polymer is taken up by mononuclear phagocyte system (MPS) organs including liver, spleen, and lung. The polymer is also detected in the lymph node after tail vein injection, which is expected due to the polymer's ability to interact with scavenger receptor A1 on endothelial cells, allowing the polymer to undergo transcytosis into the lymphatic system. This ability of the prodrug to undergo lymphatic translocation following intravenous administration appears to be unique to scavenger receptor A1 ligands, and may have tremendous therapeutic implications for infectious diseases such as HIV. Subcutaneous and intraperitoneal injections also resulted in MPS organ distribution but were not as successful at reaching lymph node, though intraperitoneal injection resulted in distribution to pancreas which may have therapeutic implications for pancreatic cancer.

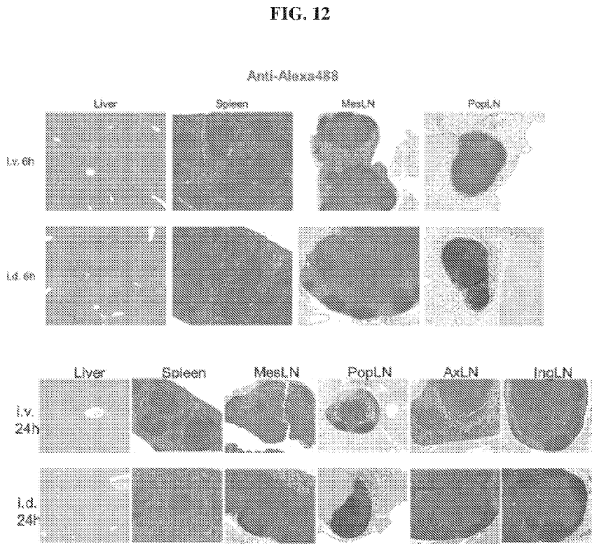

Lymph node distribution of the platform was also performed. In this study, mice were injected with polymer-488 via IV or ID administration, and several organs were harvested at 6 and 24 hours. The organs underwent tissue fixation and anti-alexa-488 staining, followed by microscopic imaging. This method allowed for high-resolution imaging of the polymer's distribution in liver, spleen, and various lymph nodes (mesenteric, popliteal, axillary, and inguinal). The resulting images showed accumulation of the prodrug platform in these tissues at both 6 and 24 hour time points (FIG. 12).

### Example 2. Allyl-Functionalized Poly(L-Lysine Succinylated)

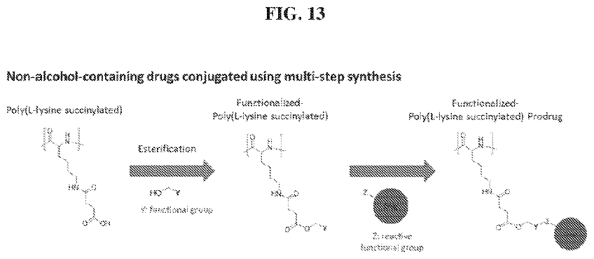

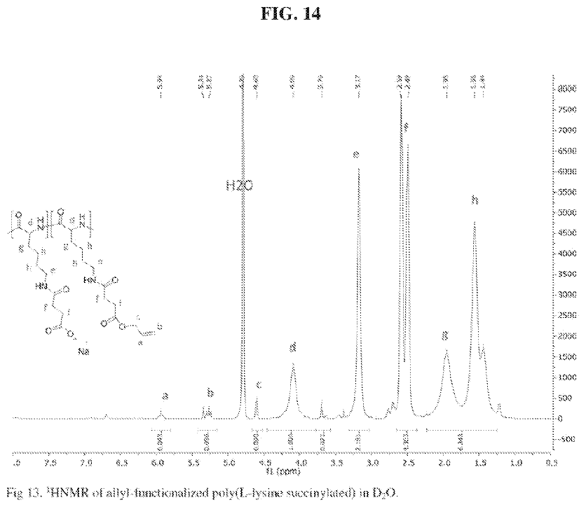

Although drugs containing alcohol groups can be conjugated directly to the polymer using a single-step esterification chemistry, additional synthetic steps are required for drugs lacking a reactive alcohol group. For example, the polymer can be modified with R—OH linkers, where OH is an alcohol that can be conjugated to the polymer using esterification and R is a carbon chain containing a reactive functional group (FIG. 13). Examples of a′ reactive functional groups include alkene, alkyne, azide, thiol, maleimide, aminooxy, ketone, aldehyde, amine, isothiocyanate, and hydrazide. Active pharmaceutical ingredients (APIs), including small molecules, peptides, proteins, oligonucleotides, and other biologics, containing reactive functional groups can be conjugated to the polymer using a specific chemistry. In an example, the poly(L-lysine succinylated) can be modified with allyl alcohol, which can then undergo thiolene chemistry with an API containing a free thiol group. The allyl-functionalized poly(L-lysine succinylated) was synthesized using esterification chemistry described for previous prodrug versions. ¹H NMR analysis confirmed allyl alcohol conjugation, and in this example there was approximately 12 allyl groups per polymer (FIG. 14).

In another example, the poly(L-lysine succinylated) is modified with an alkyne group, which then undergoes alkyne-azide chemistry with an API containing an azide group.

In another example, the poly(L-lysine succinylated) is modified with an azide group, which then undergoes alkyne-azide chemistry with an API containing an alkyne group.

In another example, the poly(L-lysine succinylated) is modified with a thiol group, which then undergoes thiolene chemistry with an API containing an alkene or maleimide group.

In another example, the poly(L-lysine succinylated) is modified with a maleimide group, which then undergoes thiolene chemistry with an API containing a free thiol group.

In another example, the poly(L-lysine succinylated) is modified with an aminooxy group, which then reacts with an API containing an aldehyde or ketone group to form an oxime bond.

In another example, the poly(L-lysine succinylated) is modified with a ketone group, which then reacts with an API containing an aminooxy group.

In another example, the poly(L-lysine succinylated) is modified with an aldehyde group, which then reacts with an API containing a hydrazide or aminooxy group.

In another example, the poly(L-lysine succinylated) is modified with an amine group, which then reacts with an API containing an isothiocyanate or NHS-ester group.

In another example, the poly(L-lysine succinylated) is modified with an isothiocyanate group, which then reacts with an API containing an amine group.

In another example, the poly(L-lysine succinylated) is modified with a hydrazide group, which then reacts with an API containing a an aldehyde group.

### Example 3. Paclitaxel Poly(L-Lysine Succinylated) Prodrug

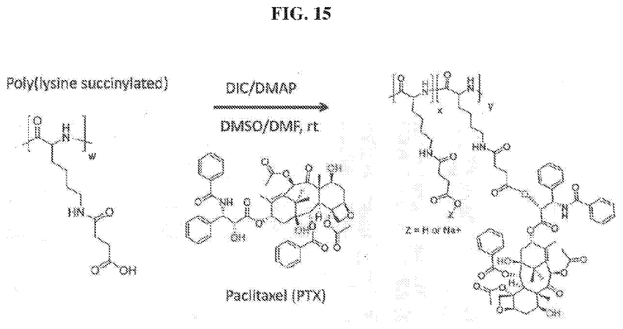

Paclitaxel was selected as a model cancer drug. Using the carbodiimide chemistry below, paclitaxel was conjugated to the poly(L-lysine succinylated) by an ester bond (FIG. 15).

Poly(L-lysine succinylated) (PLS) was converted to the free acid form by dissolving 500 mg PLS into ˜40 mL cold water and adding 2.2 mL 1N HCl. The resulting precipitant (PLS—COOH) was pelleted by centrifugation, washed several times with water, and lyophilized (yield—445 mg). PLS—COOH (385 mg, 1.69 mmol acid) and paclitaxel (75.0 mg, 0.0878 mmol) were weighed and added to an oven-dried 100 mL round-bottom flask equipped with a stir bar. The flask was capped with a rubber septum and purged with nitrogen for 5 minutes. Anhydrous DMF (19.25 mL) and anhydrous DMSO (9.625 mL) were added to the flask followed by sonication until dissolution was complete. In an oven-dried 1-dram vial, 4-dimethylaminopyridine (DMAP, 207 mg, 1.69 mmol) was added, and the vial was capped and purged with nitrogen for 5 minutes. The DMAP was then dissolved with 2.00 mL anhydrous DMSO under nitrogen. The DMAP solution was transferred to the PLS-COOH/paclitaxel reaction flask under nitrogen via syringe. N,N′-diisopropylcarbodiimide (DIC, 131 μL, 0.845 mmol) was added to the reaction flask dropwise via a microsyringe, and the reaction was allowed to stir at room temperature. The reaction was monitored using HPLC for approximately 6 hours until unreacted paclitaxel was undetectable. The reaction was then diluted with 100 mM sodium acetate buffer (pH 5.8) and dialyzed in Spectra/Por 6 regenerated cellulose dialysis tubing (10K molecular weight cut-off) against acetonitrile overnight. In order to completely remove the DMAP without cleaving the polymer prodrug product, dialysis proceeded in different solvents in the following order: 50% acetonitrile in water→sodium acetate buffer pH 5.8→100% water. Next, the product was converted to the sodium salt by raising the pH inside the dialysis bags to ˜6.3 using saturated sodium bicarbonate solution. Several rounds of dialysis against 100% water were performed at 4° C. to remove bicarbonate salts. Finally, the product was sterile filtered and lyophilized to yield a fluffy, white material (425 mg).

In this example, the prodrug was synthesized with a polymer molecular weight of 70,000 g/mol. The drug loading as high as 14.7% paclitaxel (weight to weight) has been achieved.

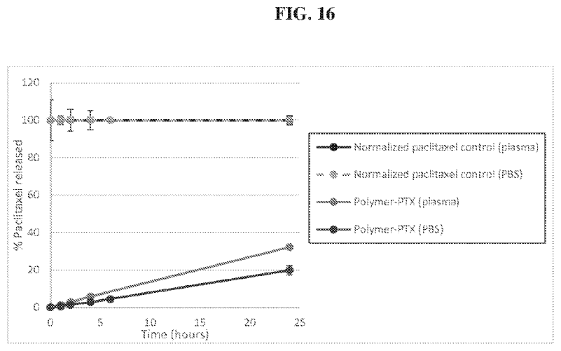

The stability and drug release of the paclitaxel polymer prodrug were assessed by incubation in fresh human plasma for 24 h. Paclitaxel was also run as a separate control to account for drug degradation due to plasma esterase activity, and a normalized drug release profile was generated to account for this degradation. The polymer prodrug demonstrated surprising stability in plasma, releasing the drug at a linear rate over time. After 24 h, about 32% of the drug had been released, after accounting for free drug degradation in plasma (FIG. 16). Drug release in PBS, which was supplemented with Tween-80 (1% v/v) to maintain solubility of released paclitaxel, was also performed to determine the extent of non-enzymatic hydrolysis of the paclitaxel polymer prodrug. Approximately 20% of the drug was released over 24 h, indicating that drug release occurs through both enzymatic and non-enzymatic mechanisms.

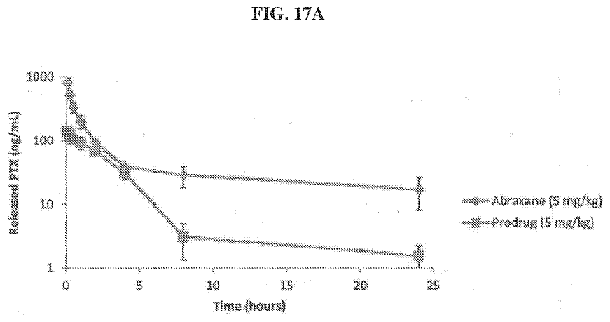

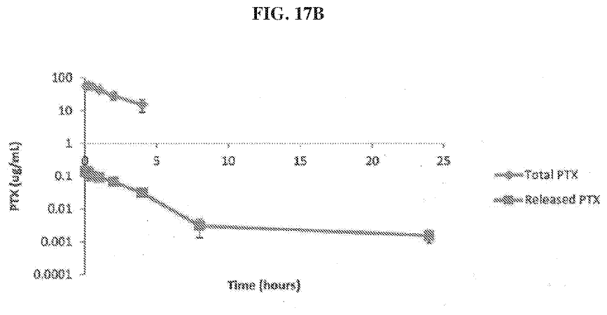

The pharmacokinetics of the paclitaxel polymer prodrug was assessed and compared to Abraxane. For each treatment group, five female Sprague-Dawley rats were dosed at 5 mg/kg, 5 mL/kg, plasma was collected at specified time points, and the released paclitaxel concentrations and total paclitaxel concentrations were measured using LC-MS/MS and LC-UV, respectively (FIGS. 17A-B). Urine was also collected at 8 and 24 hours, but no prodrug was detected in the urine. The AUC of the released paclitaxel was much lower than Abraxane. Also, the total paclitaxel concentration for prodrug group was much higher compared to free paclitaxel, indicating that the majority of paclitaxel in the plasma at any given time remains in the intact prodrug form. The prodrug is cleared fairly rapidly with a half-life of 2 hours, while the paclitaxel release half-life from the prodrug was about 40 hours, consistent with the in vitro findings. Since no prodrug was detected in urine, the pharmacokinetics can be explained by accumulation of the prodrug in tissues that express scavenger receptor A1, most likely the liver and lymphatic tissues following transcytosis across the endothelium.

As indicated above, the poly(L-lysine succinylated) paclitaxel conjugate, according to an embodiment of the present invention, releases paclitaxel in a controlled fashion with a half-life of about 40 hours in plasma, which is in sharp contrast with the drug release of the poly(glutamic acid) polymer prodrug Paclitaxel Poliglumex (Opaxio™) having a half-life of about 110 hours (Singer, J. W. et al. Paclitaxel poliglumex (XYOTAX; CT-2103): an intracellularly targeted taxane, Anti-Cancer Drug 2005, 16 (3), 243-254).

### Example 4. Lamivudine Poly(L-Lysine Succinylated) Prodrug

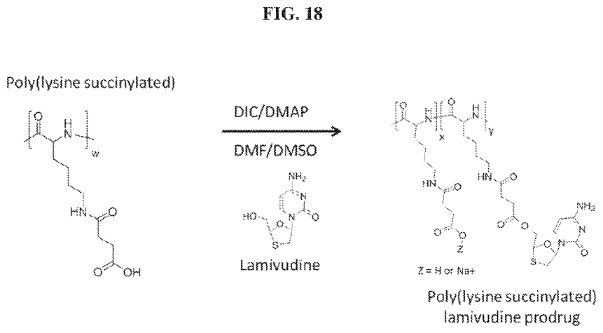

Lamivudine was selected as a model anti-HIV drug. Using the same carbodiimide chemistry described previously, the single hydroxyl group of the lamivudine is conjugated to the pendant carboxylic acid of poly(L-lysine succinylated) via an ester bond. In this example, the prodrug was synthesized with a polymer molecular weight of 70,000 g/mol (FIG. 18).

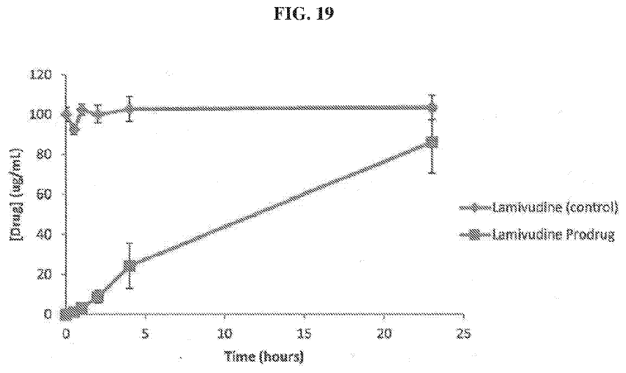

The in vitro stability and drug release of the poly(L-lysine succinylated) lamivudine prodrug were assessed in human plasma. The prodrug demonstrated linear drug release kinetics up to 24 hours with a half-life of approximately 13 hours (FIG. 19).

The present inventive concept has been described in terms of exemplary principles and embodiments, but those skilled in the art will recognize that variations may be made and equivalents substituted for what is described without departing from the scope and spirit of the disclosure as defined by the following claims.

### Example 5. Emtricitabine Poly(L-Lysine Succinylated) Prodrug

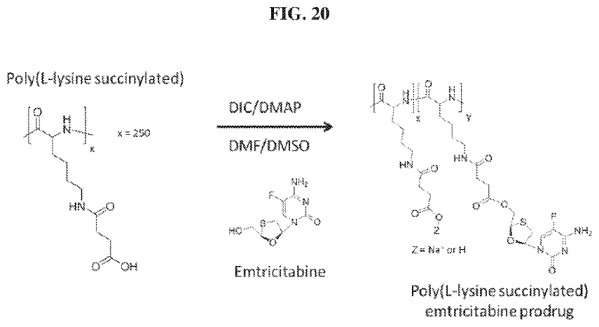

Emtricitabine was selected as an example of a clinically relevant anti-HIV drug. Using the same carbodiimide chemistry described for the prodrugs above, the single hydroxyl group of the emtricitabine was conjugated to the pendant carboxylic acid of poly(L-lysine succinylated) via an ester bond (FIG. 20).

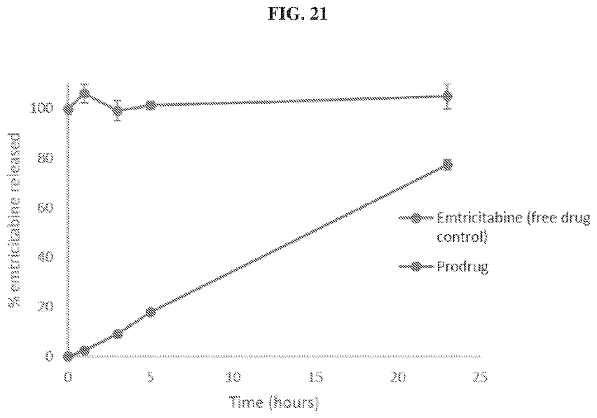

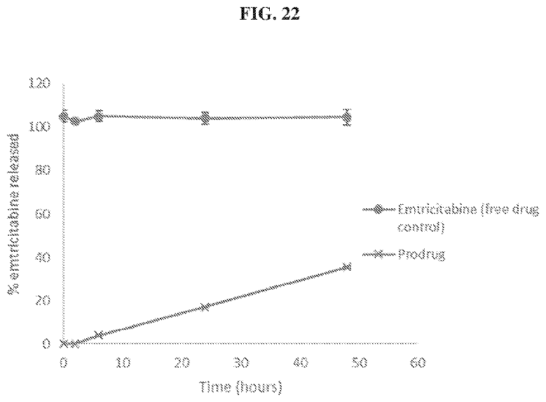

The in vitro stability and drug release of the poly(L-lysine succinylated) emtricitabine prodrug were assessed in both fresh human plasma and PBS. The prodrug demonstrated linear drug release kinetics up to 24 hours with a half-life of approximately 10 hours (FIG. 21), whereas drug release in PBS (pH 7.4) showed a drug release half-life of ˜67 hours (FIG. 22), indicating that drug release occurs through both enzymatic and non-enzymatic mechanisms.

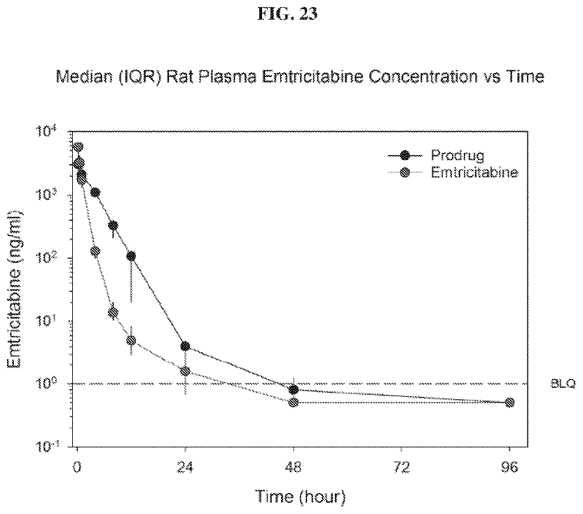

The pharmacokinetics of the emtricitabine polymer prodrug was assessed and compared to emtricitabine free drug control. For each treatment group, male Sprague-Dawley rats were dosed at 10 mg/kg emtricitabine, 10 mL/kg. At specified time points, plasma and organs were collected including liver, spleen, mesenteric, axillary, popliteal, and inguinal lymph nodes. Samples were processed and analyzed for emtricitabine concentrations using LC-MS/MS. The plasma AUC of released emtricitabine from the prodrug group was ˜2-fold higher than that of the free drug control (FIG. 23). Notably, emtricitabine concentrations in all lymph nodes from the prodrug treated animals were ˜8 to 10-fold higher than those treated with emtricitabine control, further supporting our previous claims of lymph node targeting (Table 1).

TABLE 1
NCA PK estimates for emtricitabine in tissues after IV bolus dose
at 10 mg/kg in male Sprague-Dawley rats. (LN = lymph node)

| | | | | | AUCₗₐₛₜ |
|---|---|---|---|---|---|
| Matrix | Treatment Group | Tₘₐₓ (hr) | Cₘₐₓ (ng/g) | Tₗₐₛₜ (hr) | (ng/g* hr) |
| Liver -Up Rt | Emtricitabine | 4 | 119.28 | 96 | 1095.213 |
| Lob | | | | | |
| LN Axillary | Emtricitabine | 4 | 108.66 | 96 | 918.0589 |
| LN Inguinal | Emtricitabine | 4 | 86.70 | 96 | 715.8394 |
| LN Mesenteric | Emtricitabine | 4 | 75.33 | 96 | 838.0758 |
| LN Popliteal | Emtricitabine | 4 | 105.00 | 96 | 873.3079 |
| Spleen | Emtricitabine | 4 | 106.37 | 96 | 1051.795 |
| Liver -Up Rt | Prodrug | 4 | 2256.68 | 96 | 12359.39 |
| Lob | | | | | |
| LN Axillary | Prodrug | 4 | 811.27 | 96 | 4783.154 |
| LN Inguinal | Prodrug | 4 | 586.65 | 96 | 3392.221 |
| LN Mesenteric | Prodrug | 4 | 850.66 | 96 | 4827.726 |
| LN Popliteal | Prodrug | 4 | 864.86 | 96 | 4949.075 |
| Spleen | Prodrug | 4 | 1788.13 | 96 | 10151.55 |

Other examples of anti-HIV drugs amenable to prodrug formulation include abacavir, zidovudine, ritonavir, lopinavir, atazanavir, tenofovir, and dolutegravir.

### Example 6. PI-103 Poly(L-Lysine Succinylated) Prodrug

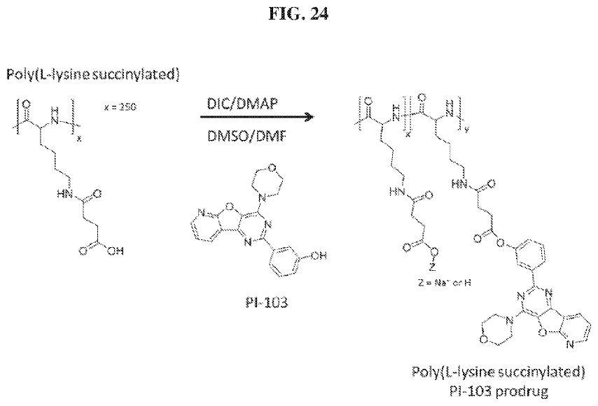

A new poly (L-lysine succinylated) prodrug version of the PI3K/mTOR dual inhibitor drug, PI-103, was developed for immunotherapy, cancer, and anti-viral indications. PI3K/mTOR inhibitors have been shown to suppress HIV through autophagy upregulation in macrophage primaries and have also been shown to promote the M2 to M1 anti-tumor polarization of tumor-associated macrophages. PI-103 was selected as a model compound, though other mTOR inhibitors, PI3K inhibitors, and dual inhibitors are amenable to this prodrug technology including PF-04691502, AZD-8055, PP-242 (torkinib), KU-0063794, PX-886, and GDC-0980 (apitolisib). The PI-103 prodrug was synthesized using esterification chemistry as described for the prodrug versions above (FIG. 24).

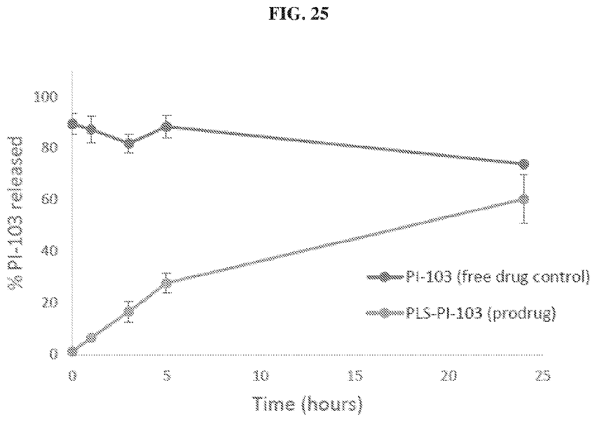

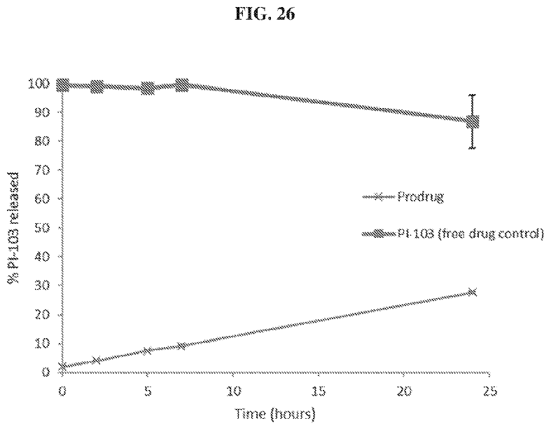

In vitro drug release in fresh human plasma demonstrated controlled-release properties, with a drug release half-life of ˜15 hours (FIG. 25). In vitro drug release in PBS (pH 7.4, 1% Tween-80 v/v) showed a drug release half-life of ˜40 hours (FIG. 26), indicating that drug release occurs through both enzymatic and non-enzymatic mechanisms.

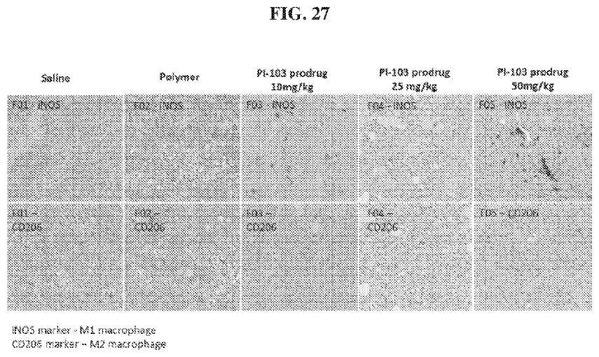

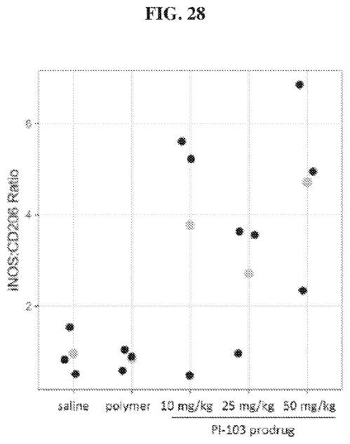

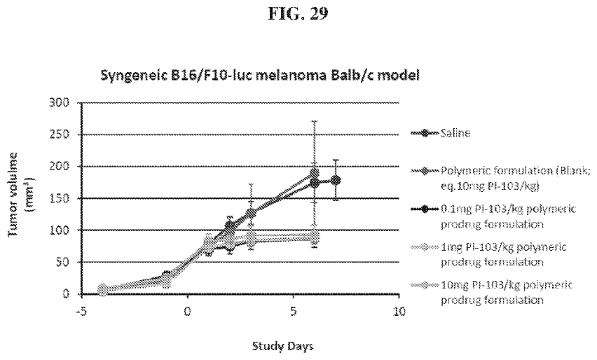

An initial MTD study was performed in a B16 F10 melanoma C57BL/6 mouse model at doses of 10, 25, 50 mg/kg PI-103 equivalent every other day for a total of five injections each. No toxicities were observed for any of the prodrug treatment groups. Additionally, the tumors were resected and fixed for M1/M2 macrophage staining and histopathology. For all prodrug treatment groups (10, 25, 50 mg/kg PI-103 equivalent), there was a marked increase in M1:M2 ratio compared to the saline and blank polymer controls (FIGS. 27, 28), indicating PI-103 reached the macrophages and induced M1 polarization as expected. In view of these results, a subsequent study in a B16 F10 melanoma model in Balb/c mice was performed. All prodrug treatment groups (0.1, 1, 10 mg/kg PI-103 equivalent, every other day for a total of five injections each) inhibited tumor growth (FIG. 29) without any loss in body weight, demonstrating anti-tumor efficacy.

Because inhibitors of mTOR were also shown to suppress Rift Valley fever virus (a zoonosis and potential bioterror threat) in animal model, the PI-103 poly(L-lysine succinylated) prodrug may also provide a targeted treatment approach against this virus, for which there is currently no standard of care.

1. A method for delivery of a therapeutically active molecule to a patient, the method comprising the steps of:
providing a composition comprising a conjugate of completely succinylated poly(lysine succinylated) and a therapeutically active molecule, and
administering the composition to a patient,
wherein the conjugate contains free/unmodified succinyl groups available for scavenger receptor A1-targeting, and
wherein the conjugate targets scavenger receptor A1; and
wherein the conjugate is represented by the following formula 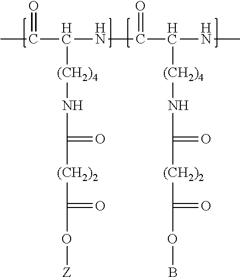
wherein Z=H or Na⁺ and B is a portion of the therapeutically active molecule, and
wherein the therapeutically active molecule is represented by a general formula B-OH.

2. The method of claim 1, wherein the therapeutically active molecule is present in a physiologically effective amount.

3. The method of claim 1, wherein the therapeutically active molecule is a small molecule drug, a peptide, or a vaccine.

4. The method of claim 1, wherein the therapeutically active molecule is an anti-cancer drug, an immunotherapy drug, or an anti-viral drug.

5. The method of claim 4, wherein the anti-cancer drug is gemcitabine, rapamycin, PI-103, PF-04691502, AZD-8055, torkinib, KU-0063794, PX-886, apitolisib, or everolimus.

6. The method of claim 4, wherein the anti-viral drug is abacavir, atazanavir, everolimus, lamivudine, emtricitabine, lopinavir, rapamycin, ritonavir, tenofovir, dolutegravir, or zidovudine.

7. (canceled)

8. The method of claim 1, wherein the composition is a controlled release composition having a drug release half-life of 3 to 80 hours.

9. A composition for delivery of a therapeutically active molecule to a patient, the composition comprising a conjugate of completely succinylated poly(lysine succinylated) and a therapeutically active molecule;
wherein the conjugate has a molecular weight of 65,000 grams per mole or greater;
wherein the conjugate contains free/unmodified succinyl groups available for scavenger receptor A1-targeting; and
wherein the conjugate targets scavenger receptor A1; and
wherein the conjugate is represented by the following formula 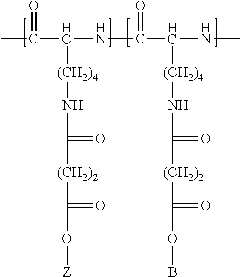
wherein Z=H or Na⁺ and B is a portion of the therapeutically active molecule, and
wherein the therapeutically active molecule is represented by a general formula B-OH.

10. (canceled)

11. (canceled)

12. The composition of claim 9, wherein the therapeutically active molecule is an anti-cancer drug, an immunotherapy drug, or an anti-viral drug.

13. The composition of claim 12, wherein the anti-cancer drug is gemcitabine, rapamycin, PI-103, PF-04691502, AZD-8055, torkinib, KU-0063794, PX-886, apitolisib, or everolimus.

14. (canceled)

15. (canceled)

16. The composition of claim 12, wherein the conjugate has the following formula: 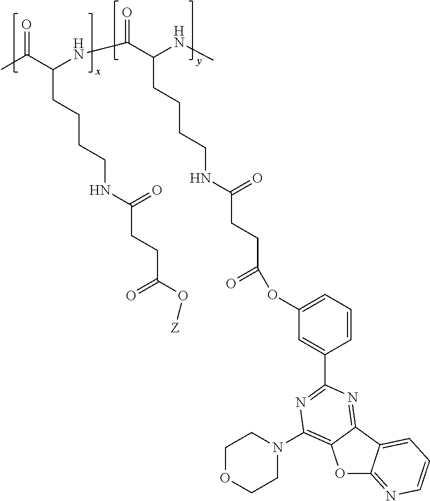
wherein, in the above formula,
x and y are variable selected such that x+y=1, and
Z is H or Na.

17. The composition of claim 12, wherein the anti-viral drug is abacavir, atazanavir, emtricitabine, everolimus, lamivudine, lopinavir, rapamycin, ritonavir, tenofovir, or zidovudine.

18. The composition of claim 17, wherein the conjugate has the following formula: 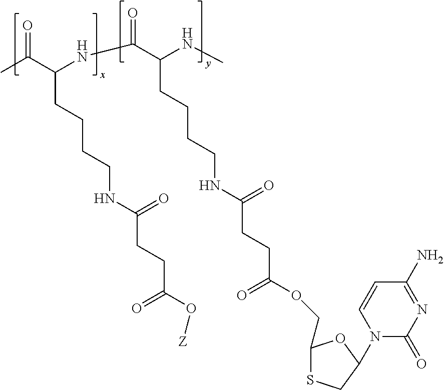
wherein, in the above formula,
x and y are variable selected such that x+y=1, and
Z is H or Na.

19. The composition of claim 17, wherein the conjugate has the following formula: 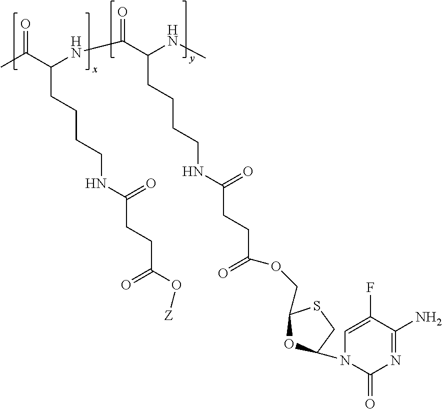
wherein, in the above formula,
x and y are variable selected such that x+y=1, and
Z is H or Na.

20. (canceled)

21. The composition of claim 9, wherein the composition is a controlled release composition having a drug release half-life of 3 to 80 hours.

22. The composition of claim 9, wherein the amount of poly(lysine succinylated) is about 85% based on the total weight of the conjugate.

23. The composition of claim 9, wherein the amount of the therapeutically active molecule is about 15% by weight based on the total weight of the conjugate.

24. The composition of claim 9, wherein a number of the therapeutically active molecules conjugated per molecule of poly(lysine succinylated) is about 10-50.

25. The composition of claim 9, wherein the composition further comprises at least one pharmaceutically acceptable excipient selected from a disintegrator, a binder, a filler, and a lubricant.

26. The composition of claim 25, wherein the disintegrator is selected from agar-agar, algins, calcium carbonate, carboxymethylcellulose, cellulose, clays, colloid silicon dioxide, croscarmellose sodium, crospovidone, gums, magnesium aluminium silicate, methylcellulose, polacrilin potassium, sodium alginate, low substituted hydroxypropylcellulose, and cross-linked polyvinylpyrrolidone hydroxypropylcellulose, sodium starch glycolate, and starch.

27. The composition of claim 25, wherein the binder is selected from microcrystalline cellulose, hydroxymethyl cellulose, and hydroxypropylcellulose.

28. The composition of claim 25, wherein the filler is selected from calcium carbonate, calcium phosphate, dibasic calcium phosphate, tribasic calcium sulfate, calcium carboxymethylcellulose, cellulose, dextrin derivatives, dextrin, dextrose, fructose, lactitol, lactose, magnesium carbonate, magnesium oxide, maltitol, maltodextrins, maltose, sorbitol, starch, sucrose, sugar, and xylitol.

29. The composition of claim 25, wherein the lubricant is selected from agar, calcium stearate, ethyl oleate, ethyl laureate, glycerin, glyceryl palmitostearate, hydrogenated vegetable oil, magnesium oxide, magnesium stearate, mannitol, poloxamer, glycols, sodium benzoate, sodium lauryl sulfate, sodium stearyl, sorbitol, stearic acid, talc, and zinc stearate.

30. The method of claim 1, wherein the amount of the therapeutically active molecule is greater than about 40% by weight based on the total weight of the conjugate.

31. The method of claim 1, wherein a number of the therapeutically active molecules conjugated per molecule of poly(lysine succinylated) is greater than about 75.

32. The composition of claim 9, wherein the amount of the therapeutically active molecule is greater than about 40% by weight based on the total weight of the conjugate.

33. The composition of claim 9, wherein a number of the therapeutically active molecules conjugated per molecule of poly(lysine succinylated) is greater than about 75.
